(19) United States
(12) Patent Application Publication
(10) Pub. No.: US 2021/0346340 A1
(43) Pub. Date: Nov. 11, 2021

(54) Pharmaceutical Compositions, Comprising a Combination of Select Carriers and Flavonoids as Antipathogens

(71) Applicant: EpitoGenesis, Inc., Sacramento, CA (US)

(72) Inventor: Shohre Golestani, Sacramento, CA (US)

(73) Assignee: EpitoGenesis, Inc., Sacramento, CA (US)

(21) Appl. No.: 17/314,239

(22) Filed: May 7, 2021

Related U.S. Application Data
(60) Provisional application No. 63/021,642, filed on May 7, 2020.

(51) Int. Cl. A61K 31/353; A61K 31/192; A61K 31/7024; A61K 31/201; A61P 31/14

(52) U.S. Cl. CPC A61K 31/353; A61K 31/192; A61P 31/14; A61K 31/201; A61K 31/7024

(57) ABSTRACT

The present invention provides flavonoid compositions that are capable of inhibiting pathogenic growth. Also provided are methods of making the compositions as well as methods of using the compositions.

### TECHNICAL FIELD

The present disclosure relates to antipathogenic and methods for using the same.

### BACKGROUND

Many existing and new disorders caused by pathogens remain untreated or undertreated by available therapies. Therefore, there exists a need for an effective, new therapeutic composition against microorganisms that cause disease or disorders.

### SUMMARY

The present invention provides flavonoid compositions that are capable of inhibiting pathogenic growth. Also provided are methods of making the flavonoid compositions as well as methods of using the flavonoid compositions.

In certain embodiments, the compositions comprise one or more cathechins.

In certain embodiments, the compositions comprise epigallocatechin gallate (EGCG).

In certain embodiments, the compositions include a pharmaceutically acceptable carrier.

In certain embodiments, the compositions include a pharmaceutically acceptable carrier; at least one flavonoid and/or at least one tannin.

In certain cases, the pharmaceutically acceptable carrier may be an organic carrier. Examples of organic carriers include liposomes, oils, lipids, fatty acids and the like. In certain embodiments, the pharmaceutically acceptable carrier may be an oil, such as animal oil, vegetable oil, fossil oil, synthetic oil, and the like. Examples of animal oil include fish oil, shark liver oil, etc. Examples of vegetable oil include mustard oil, coconut oil, safflower oil, etc.

In certain embodiments, the flavonoid is a derivative and/or salt thereof. Nonlimiting examples of the flavonoids of the invention include, a catechin, a flavonoid derivative, and a flavonoid derivative salt. In certain cases, the flavonoid is a catechin, or a derivative or a salt thereof.

In certain embodiments, the catechin is a derivative and/or salt thereof. Nonlimiting examples of the catechins of the invention include epigallocatechin gallate (EGCG), a cathechin derivative, and a cathechin derivative salt. In certain cases, the cathechin is epigallocatechin gallate (EGCG), or a derivative or a salt thereof.

In certain embodiments, the tannin is a derivative and/or salt thereof. Nonlimiting examples of the tannins of the invention include, gallic acid, a gallic acid derivative, and a gallic acid derivative salt. In certain cases, the tannin is a gallic acid, or a derivative or a salt thereof.

In certain embodiments, the composition may include a pharmaceutically acceptable carrier, a flavonoid, or a derivative or a salt thereof.

In certain embodiments, the composition may include a pharmaceutically acceptable carrier, a catechin, or a derivative or a salt thereof.

In certain embodiments, the composition may include a pharmaceutically acceptable carrier, epigallocatechin gallate (EGCG), or a derivative or a salt thereof.

The compositions may be administered to a subject, such as a mammal, by a number of routes, such as, intranasal, pulmonary, sublingual, oral, buccal, intra-vaginal, intra-rectal, ocular, intradermal, transdermal, transcutaneous, subcutaneous, intra-venous and intramuscular.

Provided are methods for making the compositions, the method includes admixing the pharmaceutically acceptable carrier and the flavonoid to produce the compositions.

Methods of using the compositions are also provided herein. The methods comprise administering the compositions to a subject to suppress or reduce that amount of pathogens or the pathogenic action of the pathogen, or reduce symptom or symptoms of disease caused by the pathogen in the subject by antipathogenic action.

In certain embodiments, the compositions are used to treat a subject with a viral infection.

In certain embodiments, the compositions are used to treat a subject infected with ssRNA viruses. Non-limiting examples of ssRNA viruses are HIV (human immunodeficiency virus), HCV (Hepatitis C virus), DENV (Dengue virus), JEV (Japanese tickborne encephalitis virus, TBEV (tickborne encephalitis virus), ZIKV (Zika virus), CIKV (Chikungunya virus), HTLV-1 (human T- cell leukemia type 1), enterovirus EV71, and PRRSV (porcine reproductive and respiratory syndrome).

In certain embodiments, the compositions are used to treat a subject with a beta-corona virus infection. Non-limiting examples of beta-corona viruses are SARS-CoV, MERS, and SARS-CoV-2.

In certain embodiments, the compositions are used to treat a subject with CoVID-19.

Before the present invention and specific exemplary embodiments of the invention are described, it is to be understood that this invention is not limited to particular embodiments described, which may, of course, vary. It is also to be understood that the terminology used herein is for the purpose of describing particular embodiments only.

Although any methods and materials similar or equivalent to those described herein can also be used in the practice or testing of the present invention, certain preferred methods and materials are now described. All publications mentioned herein are incorporated herein by reference to disclose and describe the methods and/or materials in connection with which the publications are cited.

It must be noted that as used herein, the singular forms “a”, “an”, and “the” include plural referents unless the context clearly dictates otherwise. Thus, for example, reference to “a composition” includes one and/or a plurality of such compositions, and reference to “a flavonoid” or “a tannin” includes one, two, or more flavonoids, or tannins and so forth.

### BRIEF DESCRIPTION OF THE DRAWINGS

For a more complete understanding of the present invention and the advantages thereof and by way of nonlimiting example only, reference is made to the following descriptions, taken in conjunction with the accompanying illustrative drawings, in which:

FIG. 1 is a graph showing neutralization of SARS-CoV-2 with epigallocatechin gallate (EGCG).

### DETAILED DESCRIPTION OF EXEMPLARY EMBODIMENTS

The present disclosure provides flavonoid compositions that are capable of inhibiting pathogenic growth. Also provided are methods of making the flavonoid compositions as well as methods of using the flavonoid compositions.

### Definitions

The phrase “pharmaceutically acceptable” refers to a substance that is generally safe and is acceptable for veterinary pharmaceutical use when the subject is a non-human and human pharmaceutical use, when the subject is a human.

“Penetration enhancement” or “permeation enhancement” as used herein refers to increasing the permeability of skin or mucosa to an composition so as to increase the rate at which composition antigen passes through the skin or mucosa and enters the lymph node or the blood stream.

A “therapeutically effective amount” or “efficacious amount” means the amount of a compound that, when administered to a mammal or other subject for preventing or treating a disease, is sufficient to affect such prevention or treatment for the disease. The “therapeutically effective amount” will vary depending on the compound, the disease and its severity and the age, weight, etc., of the subject to be treated.

### Overview

The present invention provides flavonoid compositions that are capable of inhibiting pathogenic growth. Also provided are methods of making the flavonoid compositions as well as methods of using the flavonoid compositions.

### Compositions

Flavonoid compositions provided herein may include: a pharmaceutically acceptable carrier; and a flavonoid.

In certain cases, the pharmaceutically acceptable carrier may be an organic carrier or an inorganic carrier. Examples of inorganic carriers include alum and other aluminum salts, e.g., aluminum hydroxide (Alum) or aluminum phosphate. Examples of organic carriers include liposomes, oils, and the like. In certain embodiments, the pharmaceutically acceptable carrier may be oil, such as, animal oil, vegetable oil, fossil oil, synthetic oil, and the like. Examples of animal oil include fish oil, shark liver oil, squalene oil, squalene, etc. Examples of vegetable oil include mustard oil, corn oil, olive oil, grape seed oil, coconut oil, safflower oil, etc. In a particularly preferred embodiment, the pharmaceutically acceptable carrier is a fish oil, such as fish squalene oil, or squalene.

In another embodiment, the compositions provided herein include: pharmaceutically acceptable oil with or without allyl isothiocyanate (essential oil of mustard); and at least one flavonoid.

In some embodiments, the compositions comprise an oil in water emulsion. The oil in water emulsion may be produced by use of a high pressure homogenization process, which applies pressures of 500-30000 psi to force the emulsion through a narrow nozzle, resulting in a homogeneous emulsion. In certain embodiments, the homogeneous emulsion comprises droplets/particle sizes of 30-100 nm.

In certain embodiments, the flavonoid compositions provided herein include: a pharmaceutically acceptable oil; and at least one flavonoid and/or tannin. In certain embodiments, the oil may be an animal oil, such as fish oil, or a vegetable oil, such as mustard oil.

In another embodiment, the compositions provided herein include: mustard oil with or without allyl isothiocyanate (essential oil of mustard); and at least one flavonoid.

In certain embodiments the pharmaceutically acceptable oil does not include oil bodies. The pharmaceutically acceptable oil may be isolated from any cell that contains oil bodies (or oil body-like structures) including plant cells, animal cells, fungal cells and bacterial cells. In certain embodiments, the pharmaceutically acceptable oil is a vegetable oil.

In the seeds of oilseed crops, which include economically important crops, such as soybean, rapeseed, sunflower and palm, the water insoluble oil fraction is stored in discrete subcellular structures known in the art as oil bodies, oleosomes, lipid bodies or spherosomes (Huang 1992, Ann. Rev. Plant Mol. Biol. 43: 177-200). Besides a mixture of oils (triacylglycerides), which chemically are defined as glycerol esters of fatty acids, oil bodies comprise phospholipids and a number of associated proteins, collectively termed oil body proteins. From a structural point of view, oil bodies are considered to be a triacylglyceride matrix encapsulated by a monolayer of phospholipids in which oil body proteins are embedded (Huang, 1992, Ann. Rev. Plant Mol. Biol. 43: 177-200). The seed oil present in the oil body fraction of plant species is a mixture of various triacylglycerides, of which the exact composition depends on the plant species from which the oil is derived.

In certain embodiments, the pharmaceutically acceptable oil of the present invention does not include substantially intact oil bodies. The term “substantially intact oil bodies” as used herein means that the oil bodies have not released greater than 50% (v/v) of their total seed oil content in the form of free oil. In certain embodiments, the pharmaceutically acceptable oil is free oil that has been released from the rupturing of the oil bodies. In certain embodiments, the pharmaceutically acceptable oil is free oil and the oil bodies present in the free oil have released greater than 50% (v/v) of their total seed oil content in the form of free oil.

In certain embodiments, the pharmaceutically acceptable oil in the compositions described herein is free oil that is prepared by a process that results in rupture of oil bodies such that the free oil does not include substantial levels of intact oil bodies. In certain embodiments, the pharmaceutically acceptable oil is prepared by a process by which 40% to 95%, such as about 45%-90%, about 50%-90%, about 60%-90%, about 70% to 90%, for example, about 40%, about 50%, about 60%, about 70%, about 80%, about 90% of the oil present in a cell is released in the form of free oil, where free oil is oil that is not present in the form of oil bodies. In other words, free oil in the form of fatty acids or triacylglycerides that is not surrounded or encapsulated by oil body proteins, such as oleosins or containing a monolayer of phospholipids . In certain embodiments, the pharmaceutically acceptable carrier is free oil which is not surrounded or encapsulated by a monolayer of phospholipids.

In certain embodiments, the pharmaceutically acceptable carrier is free oil which does not include significant levels of plant proteins, such as, proteins found in oil bodies, e.g., oil body proteins, such as, oleosin. In certain embodiments, the compositions provided herein do not include more than 0.001%-50% weight/volume (w/v) of plant protein, for example, more than about 0.001%, more than about 0.01%, more than about 0.1%, more than about 1%, more than about 10%, more than about 20%, more than about 30%, more than about 40%, more than about 50% w/v of plant protein.

In certain embodiments, the pharmaceutically acceptable carrier may be vegetable oil. The vegetable oil may be isolated from plants, such as, plant seeds. The vegetable oil may be prepared by a process by which the oil present in a plant seed is released in the form of free oil that does not include significant levels of oil bodies.

In certain embodiments, the pharmaceutically acceptable carrier may be free oil as described above. The free oil does not include significant levels of oil bodies. In certain embodiments, the free oil does not include more that 0.0000001% weight/volume to 50% weight/volume of oil bodies. In certain cases, the oil bodies are present at less than 50% weight/volume, less than 40% weight/volume, less than 30% weight/volume, less than 20% weight/volume, less than 10% weight/volume, less than 5% weight/volume, less than 1% weight/volume, less than 0. 5% weight/volume, less than 0.1% weight/volume in the free oil present in the compositions described herein.

In certain embodiments, the free oil present in the compositions provided herein does not include more than 0.001%-50% weight/volume (w/v) of plant protein, e.g., oil body protein, for example, more than about 0.001%, more than about 0.01%, more than about 0.1%, more than about 1%, more than about 10%, more than about 20%, more than about 30%, more than about 40%, more than about 50% w/v of plant protein.

The flavonoid comprises flavonoid derivatives, salts and salts of derivatives. In certain embodiments, the flavonoid is a flavone, a flavonol, a flavonone, a catechin, anthocyanid, or isoflavone, or derivatives, salts, or salts of the derivatives thereof. In certain embodiments, the flavonoid is a catechin, such as, catechin hydrate. In certain embodiments, the flavonoid is a epigallocatechin gallate (EGCG).

The compositions may additionally include other additives, such as preservatives, colorants, flavorants, etc.

### Pharmaceutically Acceptable Organic Carriers

A “pharmaceutically acceptable vegetable oil carrier” as used herein refers to a vegetable oil that is suitable for administration to a human or non-human animal by a desirable route, e.g., systemic or mucosal route, including oral and topical routes of delivery. Edible compositions are contemplated by the present disclosure.

“Vegetable oil” refers to oil obtainable from a plant or a plant product, and encompasses oil obtainable from seeds (including nuts, grains), fruits, roots, flowers, stems, etc. Examples include corn oil, mustard oil, olive oil, grape seed oil, coconut oil, safflower oil, soybean oil, squalene oil or squalene, and the like. Vegetable oils of the present disclosure encompass oils obtainable from non-genetically modified and from genetically modified plants. Vegetable oils encompass vegetable oils having properties of a rubefacient, i.e., oils that promotes dilation of capillaries and an increase in blood circulation, e.g., when applied topically to skin. Vegetable oil may be derived from a plant or plant product (e.g., a non-genetically modified or genetically modified plant or plant product), or may be produced synthetically, e.g., by mixing the individual components found in vegetable oils, where the individual components may be derived from any source, such as, plants or plant products, animals, animal products, fossil oils, or produced synthetically. The plants which provide the source for the vegetable oil or the individual fatty acids may be genetically modified.

In certain embodiments, the vegetable oil is a mustard oil. “Mustard oil” as used herein refers to oil that is obtainable from seeds of a mustard plant of Brassicacae, where the oil is obtainable from the mustard plant without application of heat during extraction (e.g., obtainable by a cold-press extraction method). Mustard oil obtainable from seeds of a mustard plant without application of heat have a lower amounts of (e.g., no significant or detectable) allyl isothiocyanate than oil that may be obtainable from the same seeds using a heat-based extraction method (e.g., by application of steam). Mustard plants of Brassicacae from which mustard oils useful as carriers in the compositions of the present disclosure may be obtainable include, but are not necessarily limited to, Brassica rapa (edible greens), Brassica nigra (black mustard), Brassica juncea (brown mustard), Brassica hirta (white or yellow mustard), Brassica carinata (Ethiopian mustard), Brassica oleracea (wild mustard), Brassica campestris (including Brassica napus L. and B. campestris L.), and Brassica napus. Oils contemplated by “mustard oil” can include oil obtainable from rapeseed.

As noted in the preceding section, the vegetable oil is preferably free oil and as such does not comprise substantial levels of substantially intact oil bodies. In certain embodiments, the vegetable oil is canola oil. Such canola oil may have the following composition: 6-8% Saturated Fatty Acids (with 3.5 Palmitic Acid); 14.4% Monounstaurated Fatty Acids (with 60% Oleic Acid); and 69.3% Polyunsaturated Fatty Acids (with 20% Linoleic Acid, 10% Alpha Linolenic Acid).

In certain embodiments, the vegetable oil used in the compositions described herein may comprise about 14%-70% monounsaturated fatty acids, about 18%-22% polyunsaturated fatty acids and about 5%-12% saturated fatty acids. The monounsaturated fatty acids may have about 18%-51% erucic acid and about 7%-22% oleic acid, the polyunsaturated fatty acids may have about 9-15% linolenic acid and about 6-24% linoleic acid, and the saturated fatty acids may have about 3-4% palmitic acid.

In certain embodiments, the vegetable oil used in the compositions described herein may comprise about 14%-70% monounsaturated fatty acids, 18%-22% polyunsaturated fatty acids and 5%-12% saturated fatty acids.

In certain embodiments, the vegetable oil used in the compositions described herein may comprise about 14%-20% monounsaturated fatty acids, 18%-20% polyunsaturated fatty acids and 5%-6% saturated fatty acids.

In certain embodiments, the vegetable oil used in the compositions described herein may comprise about 60%-70% monounsaturated fatty acids, about 18%-22% polyunsaturated fatty acids and about 5%-6% saturated fatty acids.

Where the vegetable oil is a mustard oil, in certain embodiments, the mustard oil may have the following composition: monounsaturated fatty acids (erucic acid (18-51%), oleic acid (7-22%)), polyunsaturated fatty acids (linolenic (9-15%) and linoleic (6-24%)), and 5% saturated fatty acids. The mustard oil may additionally also include other components, such as, proteins (30%), phenolics, phytin and dithiol thiones. Mustard oil may also contain 490 mg/100 gm of calcium. Mustard oil may also contain 9-15% omega 3 fatty acids.

In some embodiments, the mustard oil is one obtainable from Brassica rapa. Mustard oil obtainable from Brassica rapa includes an oil having the following composition: 5.4% Saturated Fatty Acids (with 2.7% Palmitic Acid, 1.0% Stearic Acid, 0.6% Behenic, and 1.1% other saturated fatty acids); 67.3% Monounsaturated Fatty Acids (with 23.3% Oleic, 10.0% Gadoleic, 33.8% Erucic); and 20.6% Polyunsaturated Fatty Acids (with 9.4% Linoleic Acid, 9.9% Alpha Linolenic Acid).

The present disclosure also contemplates compositions having a vegetable oil carrier that itself is a rubifacient and/or combined with a rubefacient oil. Examples of rubefacient oils include Oil of Wintergreen (Methyl Salicylate), mustard oil, and Rosemary oil (Rosmarinus officinalis).

In other embodiments, the oil carrier may be a single fatty acid (e.g. oleic acid) or combinations of two or more fatty acids.

In certain embodiments, the pharmaceutically acceptable carrier may be oil in the form of fatty acids, such as omega 3 (e.g. eicosapentaenoic acid (EPA), and docosahexaenoic acid (DHA)) or omega-6 fatty acids (e.g. linoleic acid), in various proportions, e.g. 1:1, isolated from plant or animal oils or genetically modified microorganisms or produced by chemical synthesis

In certain embodiments the vegetable oil may contain or be solely squalene from vegetables including but not limited to amaranth seed, rice bran, wheat germ, and olive.

### Pharmaceutically Acceptable Animal Oil Carriers

In certain embodiments, the pharmaceutically acceptable carrier may be an oil, such as an animal oil. Animal oils include oils derived from an animal source or synthesized from individual fatty acids and mixed to produce an oil similar to animal oil. Examples of animal oils include fish oil, shark liver oil, cod oil, animal squalene, butter, chicken fat, lard, dairy butterfat, or combinations thereof, and the like. In a preferred embodiment, the pharmaceutically acceptable carrier comprises fish oil.

### Pharmaceutically Acceptable Fossil Oil Carriers

In certain embodiments, the pharmaceutically acceptable carrier may be an oil, such as a fossil oil. In certain embodiments, the pharmaceutically acceptable carrier may be mineral oil. Mineral oil or liquid petroleum is a by-product in the distillation of petroleum to produce gasoline and other petroleum based products from crude oil. Mineral oil is composed mainly of alkanes (typically 15 to 40 carbons) and cyclic paraffins, related to petroleum jelly (also known as “white petrolatum”). It has a density of around 0.8 g/cm³. Mineral oil is available in light and heavy grades, and can often be found in drug stores. There are three basic classes of refined mineral oils: paraffinic oils, based on n-alkanes; naphthenic oils, based on cycloalkanes; and aromatic oils, based on aromatic hydrocarbons.

### Other Pharmaceutically Acceptable Carriers

It is contemplated that the carrier of the invention can be any suitable pharmaceutically acceptable carrier. In certain embodiments the pharmaceutically acceptable carrier may be virosomes, liposomes, or ISCOMS.

### Flavonoids

The compositions may include one or more flavonoids or derivates, salts or salts of derivatives thereof. Flavonoids (also known as bioflavonoids) are phytochemicals found in fruits and vegetables. Flavonoids are of the following types: Flavones (e.g., apigenin, luteolin), Flavonols (e.g., quercetin, myricetin), Flavanones (e.g., naringenin, hesperidin), Catechins (e.g., epicatechin, catechin, gallate, such as, epigallocatechin, gallocatechin, epicatechin gallate and epigallocatechin gallate), Anthocyanidins/anthocyanins (e.g., cyanidin, pelargonidin), and Isoflavones (e.g., genistein, daidzein).

In a preferred embodiment, the flavonoid is a catechin. In certain embodiments, the compositions may include epigallocatechin gallate (EGCG), a form of catechin (polyphenols). In some embodiments, the compositions may include a catechin, such as, catechin hydrate. In some embodiments, the catechin is not a multimeric form of catechin.

In certain cases, the \ compositions may include EGCG derivatives, such as those described in U.S. Pat. No. 7,544,816.

In certain embodiments, the compositions may include phytochemicals, such as flavonoids, and analogues thereof, such as those described in U.S. Pat. No. 7,601,754.

### Tannins

Tannins are a subclass of plant derived polyphenols and have a high binding affinity for proteins. “Tannin” is a general descriptive name for a group of polymeric phenolic substances capable of tanning leather or precipitating gelatin from solution, a property known as astringency. Their molecular weights range from 500 to 3,000, and they are found in almost every plant part: bark, wood, leaves, fruits, and roots. They are divided into two groups, hydrolyzable and condensed tannins. Hydrolyzable tannins are based on gallic acid, usually as multiple esters with D-glucose, while the more numerous condensed tannins (often called proanthocyanidins) are derived from flavonoid monomers. Tannins may be formed by condensations of flavan derivatives which have been transported to woody tissues of plants. Alternatively, tannins may be formed by polymerization of quinone units. One of the molecular actions of tananins is to complex with proteins through so-called nonspecific forces such as hydrogen bonding and hydrophobic effects, as well as by covalent bond formation. Thus, their mode of antimicrobial action may be related to their ability to inactivate microbial adhesins, enzymes, cell envelope transport proteins, etc. They also complex with polysaccharide. The antimicrobial significance of this particular activity has not been explored (Clinical Microbiology Reviews; Oct. 1999, vol. 12; p. 564-582). Variable immune responses to tannins has stunted research into the properties of these plant metabolites. Increasing evidence demonstrates select binding affinities of individual tannin species that explains, in part, the discrepancies in immunological function. Gamma-delta TCR+T cells can be activated by a select group of tannins called procyanidins (also called condensed tannins) (Crit Rev Immunol. 2008; 28(5): 377-402. Response of gammadelta T Cells to plant-derived tannins. Holderness J, Hedges J F, Daughenbaugh K, Kimmel E, Graff J, Freedman B, Jutila M A). Structurally, tannins are divided into gallotannins, Ellagitannins, complex tannins, and condensed tannins. (1) Gallotannins are all those tannins in which galloyl units or their meta-depsidic derivatives are bound to diverse polyol-,catechin-, or triterpenoid units. (2) Ellagitannins are those tannins in which at least two galloyl units are C—C coupled to each other, and do not contain a glycosidically linked catechin unit. (3) Complex tannins are tannins in which a catechin unit is bound glycosidically to a gallotannin or an ellagitannin unit. (4) Condensed tannins are all oligomeric and polymeric proanthocyanidins formed by linkage of C-4 of one catechin with C-8 or C-6 of the next monomeric catechin. Tannin examples include but are not limited to: tannic acid, gallica acid, (-)-Epigallocatechin gallate (EGCG), (-)-epicatechin gallate (ECG), Resveratrol, piceatannol, geraniin, pedunculagin and corilagin. Acertannin, Hamamelitannin, (Nat. Prod. Rep., 2001, 18, 641-649).

### Additives

In certain embodiments, the vegetable oil carrier of the composition may include allyl isothiocyanate (AIT), as an additive at the preferred dose of 0.1-2% of the final dose volume. Allyl isothiocyanate (AIT) is also referred to as volatile oil of mustard or essential oil of mustard or oil of mustard. AIT is an organosulfur compound of the formula CH₂CHCH₂NCS. AIT is responsible for the pungent taste of mustard, horseradish, and wasabi. It is slightly soluble in water, but well soluble in most organic solvents. Allyl isothiocyanate comes from the seeds of black or brown Indian mustard. When these mustard seeds are broken, the enzyme myrosinase is released and acts on a glucosinolate known as sinigrin to give allyl isothiocyanate. Allyl isothiocyanate serves the plant as a defense against herbivores; since it is harmful to the plant itself, it is stored in the harmless form of the glucosinolate, separate from the myrosinase enzyme. When an animal chews the plant, the allyl isothiocyanate is released, repelling the animal. Allyl isothiocyanate is produced commercially by the reaction of allyl chloride and potassium thiocyanate: CH₂=CHCH₂Cl+KSCN→CH₂=CHCH₂NCS+KCl. The product obtained in this fashion is sometimes known as synthetic mustard oil. Allyl isothiocyanate can also be liberated by dry distillation of the seeds. The product obtained in this fashion is known as volatile oil of mustard and is usually around 92% pure. It is used principally as a flavoring agent in foods. Synthetic allyl isothiocyanate is used as an insecticide, bacterialcide, and nematocide, and is used in certain cases for crop protection.

The compositions may include other additives or carriers, such as, gelatin, antibiotics, sorbitol, sucrose, lactose, other sugars, bioadhesives, mucoadhesives (e.g., hyaluronic acid or a derivative thereof, for example, HYAFF), hydrophilic polymers and hydrogels, polyethylene oxide homopolymers, chitosan, Beeswax, and the like.

In certain embodiments, where the pharmaceutically acceptable carrier is oil-based and the composition comprises an oil based emulsion. In such embodiments, the oil-based emulsion may not include organic phosphates, such as those used in phosphate buffered saline (PBS).

The compositions may further include emulsifiers, such as, lecithin, for example phospholipids and/or surfactants that are amphiphilic and acceptable for human and/or veterinary use.

Surfactants are well known to the skilled artisan, and include, interalia, ionic surfactants (e.g. Tween 80), cationic surfactants (e.g. CTAB) or zwitterionic surfactants (e.g. CHAPS). The acceptability of a surfactant for human and/or veterinary use may be determined by those of skill in the art. A surfactant is amphiphilic if a part of the surfactant molecule is hydrophobic and a part is hydrophilic. Examples of surfactants useful in the compositions disclosed herein include, but are not limited to, a Tween surfactant and a Span surfactant. Tween and Span surfactants include, but are not limited to, monolaureate (Tween 20, Tween 21, Span 20), monopalmitate (Tween 40, Span 40), monostearate (Tween 60, Tween 61, Span 60), tristearate (Tween 65, Span 65), monooleate (Tween 80, Tween 81, Span 80) and trioleate (Tween 85, Span 85).

The compositions may include pharmaceutically acceptable auxiliary substances as required to approximate physiological conditions such as pH adjusting and buffering agents, and the like, for example, sodium acetate, sodium chloride, potassium chloride, calcium chloride, sodium lactate, phosphate buffer saline, and the like.

The compositions may include medicinal rubefacients, such as, Capsaicin (derived from Cayenne, Capsicum minimum), Salicylates (such as Oil of Wintergreen, Methyl Salicylate), Nicotinate esters, Rubbing alcohol, common herbal rubefacients include: Cloves (Eugenia caryphyllus), Garlic (Allium sativum), Ginger (Zingiber officinale), Horseradish (Cochlearia armoracia), Mustard (e.g., Brassica alba or B. nigra), Nettle (Urtica dioica), Rosemary Oil (Rosmarinus officinalis), Rue (Ruta graveolens).

### Method of Making Composition

Provided herein are methods of making the flavonoid compositions. The methods comprise admixing a pharmaceutically acceptable carrier and a flavonoid to produce the compositions.

The components of the subject compositions may be obtained from a variety of sources using a number of methods. Alternatively, the components may be synthesized chemically. In certain cases, the components may be isolated from a natural source and may be additionally modified, e.g., chemically modified. For example, mustard oil may be extracted from mustard plant seeds. Alternatively, the parmaceutically acceptable vegetable oil or animal oil carrier may be purchased from a vendor. The flavonoids, e.g., catechins, for example, catechin hydrate, may be purchased from Sigma Aldrich chemical company, prepared and produced by standard biochemical methods.

In general, Catechins may either be extracted from green tea or synthesized chemically. Korean and Chinese green tea, and pu-erh, Indian black, Longjing, Tieguanyin, Bamboo, Jasmine, Oolong, Flower, Red teas may be used for extracting catechins, such as, epigallocatechin, catechin, epicatechin, epigallocatechin gallate and epicatechin gallate. Chinese green tea is a rich source of catechin. Green tea is a better source of catechin compared to the other types of tea.

In certain embodiments, a pharmaceutically acceptable oil and a flavonoid and/or a tannin may be mixed together in amounts as described above along with a surfactant such as Tween®-80. Before administrating, the composition may be emulsified by repeatedly withdrawing and releasing the mixture of a pharmaceutically acceptable oil, a surfactant(s), and another component(s).

In certain embodiments, a pharmaceutically acceptable organic or inorganic carrier may be mixed with watersoluble flavonoids, and tannins.

The components of the compositions may be sterilized prior to admixing or after forming the compositions. The compositions may be mixed with a gel, or formulated into microparticles, etc. before administration.

The compositions disclosed herein may be formulated into a spray (e.g., nasal or pulmonary spray), drops (e.g., nasal drops), gel, powder, tablets or capsules, patch, and the like. Of particular interest are compositions suitable for administration via inhalation including but not limited to, liquid suspensions for forming aerosols as well as powder forms for dry powder inhalation delivery systems. Devices suitable for administration by inhalation of subject composition include, but are not limited to, atomizers, vaporizers, nebulizers, and dry powder inhalation delivery devices.

The compositions disclosed herein may be formulated into liquids or emulsions. In the course of the formulation process any type of emulsion may be formed, including without limitation an oil-in-water emulsion, a water-in-oil emulsion, a multiple (e.g. double, tri-multiple, quarter-multiple, etc.) emulsion, and reverse emulsion. The compositions of the present invention may be in the form two phases where one phase is uniformly dispersed in the other phase, resulting in a homogenous macroscopic appearance. Where compositions comprising two or more non-uniformly dispersed phases are formed, the phases may be shaken or stirred prior to use of the emulsion. In certain embodiments, as noted above, oil-in-water emulsions may be produced by use of a high pressure homogenization process, which applies pressures of 500-30000 psi to force the emulsion through a narrow nozzle, resulting in a homogeneous emulsion with droplets/particle sizes of 30-100 nm.

In certain embodiments, the compositions provided herein do not include a solubilizing agent as described in United States Patent Application No. 20080254188. In certain embodiments, the compositions described herein are not water-slouble formulations, rather, they are water insoluble formulations, such as, emulsions. The term water-soluble means that the formulation when added to an aqueous medium (e.g., water) dissolves in the aqueous medium to produce a solution that is essentially clear. In one example, the formulation dissolves in the aqueous medium without heating the resulting mixture above ambient temperature (e.g., 25° C.). Essentially clear means that the composition is transparent and essentially free of visible particles and/or precipitation (e.g., not visibly cloudy, hazy or otherwise non-homogenous).

### Method of Using Compositions

The present disclosure provides methods for inhibiting or reducing pathogenic growth. The compositions disclosed herein can be useful for prophylaxis, prevention, and/or treatment of various infections.

### Conditions

In certain embodiments, the compositions disclosed herein may find use in the context of administering an antipathogen, such as an antiviral.

The compositions may be used to treat a subject infected with ssRNA viruses. Non-limiting examples of ssRNA viruses are HIV (human immunodeficiency virus), HCV (Hepatitis C virus), DENV (Dengue virus), JEV (Japanese tickborne encephalitis virus, TBEV (tickborne encephalitis virus), ZIKV (Zika virus), CIKV (Chikungunya virus), HTLV-1 (human T-cell leukemia type 1), enterovirus EV71, and PRRSV (porcine reproductive and respiratory syndrome).

The compositions may be used to treat a subject with a beta-corona virus infection. Non-limiting examples of beta-corona viruses are SARS-CoV, MERS, and SARS-CoV-2. The compositions may be used to treat a subject with CoVID-19, the disease caused by SARS-CoV-2.

### Route of Administration

The compositions disclosed herein may be administered to a subject via a number of routes of administration. Exemplary routes of administration include mucosal, e.g., oral, sublingual, intra-nasal, inhalation, ocular, intra-vaginal, intra-rectal; and systemic, e.g., intra-muscular, intra-dermal, trans-dermal, intraperitoneal, subcutaneous or trans-cutaneous. In certain embodiments, a combination of at least two routes of administration may be used. For example, a combination of a mucosal route and a systemic route of administration may be used.

The route of administration may vary based on the individual subject and the stage of the disease and other factors evident to one skilled in the art.

When the route of administration is a mucosal or trans-epithelial (through the skin) route, compositions comprising allyl isothiocyanate or wintergreen are preferred.

In certain embodiments, the compositions described herein may be administered through the mucosal surface without breaking the mucosal surface.

The compositions disclosed herein may be provided as micro- or nano-particles in gel or tablet (such as, fast dissolving) forms. Such formulations may be administered via oral or sublingual routes, for example. For intra-nasal administration, the compositions may be formulated as nasal sprays in an emulsion form or drops, for example. For transcutaneous administration, compositions may be given in a gel, lotion or ointment form. For systemic injections, the compositions can be given formulated as an emulsion and/or micro/nanoparticles. For rectal administration, the compositions can be formulated as suppository or gels, for example. For vaginal administration, the compositions formulated as gel, emulsion, ointment, for example.

In certain embodiments, the compositions disclosed herein may be administered to a subject via a combination of different routes in the order indicated below:

i. systemic, mucosal;

ii. systemic, systemic, mucosal, mucosal;

iii. systemic, mucosal, systemic;

iv. mucosal, mucosal, systemic, systemic;

v. mucosal, systemic, systemic;

vi. mucosal, systemic, mucosal, for example.

When an composition is administered systemically or mucosally more than once, the two or more systemic or mucosal administrations may be by the same systemic (for example, two intramuscular injections) or mucosal route (two intra-nasal (IN)/sublingual (SL) administrations) or different (for example, one intramuscular injection and one intravenous injection; one IN administration and one SL administration).

### Dosages

The dosage of the compositions described herein to be administered to a subject comprising may be determined based on the route of administration and body weight and may range from 0.001 mg/kg body weight to 100 mg/kg body weight. The number of times an composition is administered may vary and may be determined based upon numerous factors. These factors are evident to a person of skill in the art.

### Subjects

The compositions described herein may be used combat in infection. In certain cases, the compositions described herein may be administered to any member of the subphylum chordata, including, mammals (humans, other non-human primates, domesticated animals, e.g., livestock), avians, fishes, or any other animal in need thereof. In certain cases, the compositions may be administered to humans. In certain cases, the compositions may be administered to cows. In certain cases, the compositions may be administered to chickens, horse, sheep, goats. In certain cases, the compositions may be administered to porcines. In certain cases, the compositions may be administered to cats and dogs.

### Kits

Kits that include one or more sterile containers of components of the compositions described herein are also provided. Individual components may be present in separate sterile containers or two or more components may be present in a single container. Optionally, the kit may also include a container containing a desired flavonoid.

In some embodiments, the sterile containers may optionally have an access port(s) for withdrawing a specific volume/amount of a component, for example, a port for introducing a syringe to withdraw a certain volume of a pharmaceutically acceptable carrier.

In some embodiments, the containers of the components of the compositions described herein may not be sterile but are reasonably clean.

The kits may further include a suitable set of instructions, generally written instructions, relating to the use of the composition as an antipathogenic agent.

The kits may comprise the components of the composition packaged in any convenient, appropriate packaging. For example, if a component is a dry formulation (e.g., freeze dried or a dry powder), a vial with a resilient stopper may be used, so that the component may be easily resuspended by injecting fluid through the resilient stopper. Ampoules with non-resilient, removable closures (e.g., sealed glass) or resilient stoppers may be used for liquid component(s) of the composition. Also contemplated are packages for use in combination with a specific device, mucosal administration devices, such as, an inhaler, nasal administration device (e.g., an atomizer) or eye drops.

The instructions relating to the use of composition generally include information as to dosage, dosing schedule, and route of administration. The containers of containing the components of composition or the premixed composition may be unit doses, bulk packages (e.g., multi-dose packages) or sub-unit doses. Instructions supplied in the kits disclosed herein are typically written instructions on a label or package insert (e.g., a paper sheet included in the kit), but machine-readable instructions (e.g., instructions carried on a magnetic or optical storage disk) may also be included.

### EXAMPLES

### Example 1: In Vitro Determination of the Antiviral Activity of EGCG to Neutralize SARS-CoV-2

A neutralization assay with epigallocatechin gallate (EGCG) is performed and quantified using an Avicel plaque assay.

### Culture

VeroE6 cells (BEI Resources) are maintained in Dulbecco's modified Eagle medium (DMEM) with 10% heat inactivated fetal bovine serum, GlutaMAX, non-essential amino acids and sodium pyruvate. The day prior to the neutralization assay, VeroE6 cells will be seeded at 600,000 cells per well of a 6-well plate in 2 mL maintenance media. Virus was propagated in DMEM as previously described, with the exception of 2% heat inactivated fetal bovine serum.

### Viral Propagation

Passage 4 SARS-CoV-2 USA-WA1/2020 was received from the University of Texas Medical Branch. A T225 flask of VeroE6 cells was inoculated with 90 μL starting material in 15 mL DMEM/2% HI-FBS. A control flask was mock-infected at the same time. Both flasks were incubated in a humidified incubator at 37° C./5% CO2 with periodic rocking for 1 hour. After 1 hour, 60 mL of DMEM/2% HI-FBS was added to each flask without removing the inoculum and flasks were incubated again at 37° C./5% CO₂. Flasks were observed daily for progression of CPE and stock was harvested at 66 hours post-inoculation (as previously determined based on published data and prior experience with SARS-CoV). Stock supernatant was harvested and clarified by centrifugation at 5,250 RCF at 4° C. for 10 minutes and heat inactivated fetal bovine serum concentration was increased to 10% final concentration. Neutralization Assay: All viral infection quantification assays will be performed at biosafety level 4 (BSL-4) at the National Emerging Infectious Disease Laboratories (NEIDL). An Avicel plaque reduction assay will be used to quantify plaques. In brief, EGCG will be serially diluted 2-fold from 500 μg/ml to 0.98 μg/ml. Each EGCG dilution will be mixed with 100 PFU/mL SARS-CoV-2 for 1 hour at 37° C. The maintenance media will be removed from each plate and 200 μl of inoculum will be added. The plates will then be incubated for 1 hour at 37° C./5% CO2 with intermittent rocking. The overlay will be prepared by mixing by inversion Avicel 591 overlay and 2× plaque assay media in a 1:1 ratio. After 1 hour, the inoculum will then be removed, and 2 ml overlay will be added to each well and the plates will be incubated for 48 hours at 37° C./5% CO₂. 6-well plates will then be fixed using 10% neutral buffered formalin prior to removal from BSL-4 space. The fixed plates will then be stained with 0.2% crystal violet in 10% neutral buffered formalin and the plaques counted.

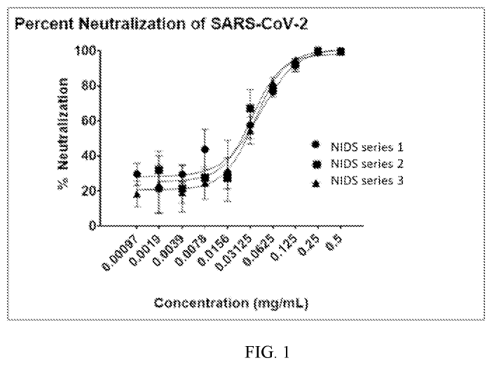

FIG. 1 shows a strong in vitro antiviral activity of EGCG on vero cells infected with SARS-CoV-2. An Avicel plaque reduction assay was used to quantify plaques. EGCG was serially diluted 2-fold from 500 μg/ml to 0.98 μg/ml. Each EGCG dilution was mixed with 1,000 PFU/ml (100 PFU/well) SARS2 for 1 hour at 37° C. Each dilution series of EGCG/SARS2 was done in triplicate (dilution series 1-3) with three replicates and the following IC50 were recorded: Dilution series 1—42.65 μg/ml, dilution series 2—32.89 μg/ml and dilution series 3—36.09 μg/ml. The results were normalized against the control (virus in DMEM). Any wells which were obscured in anyway were removed and not used as a datapoint. All viral infection quantification assays were performed at biosafety level 4 (BSL-4) at the National Emerging Infectious Disease Laboratories (NEIDL). These data demonstrate the relatively strong antiviral activity of EGCG.

1. An antimicrobial or antiviral composition comprising a flavonoid and a pharmaceutically acceptable carrier.

2. The antimicrobial or antiviral composition according to claim 1, wherein the flavonoid is a catechin.

3. The antimicrobial or antiviral composition according to claim 1, wherein the flavonoid is epigallocatechin gallate (EGCG).

4. The antimicrobial or antiviral composition of claim 1, wherein the composition comprises a tannin.

5. The antimicrobial or antiviral composition of claim 1, wherein the tannin is gallic or tannic acid.

6. The antimicrobial or antiviral composition according to claim 3, comprising from about 5% to about 50% oleic acid.

7. The antimicrobial or antiviral composition according to claim 6, comprising 40% oleic acid.

8. The antimicrobial or antiviral composition according to claim 3, comprising the catechin or tannin in liposomes or substances that increase the bioavailability of the tannin or catechin.

9. A method of treating an infection in a mammalian subject, comprising administering to said subject an antimicrobial or antiviral composition according to claim 3.

10. The antimicrobial or antiviral composition according to claim 7, comprising the catechin or tannin in liposomes or substances that increase the bioavailability of the tannin or catechin.

11. A method of treating an infection in a mammalian subject, comprising administering to said subject an antimicrobial or antiviral composition according to claim 3.
